(19) United States
(12) Patent Application Publication
(10) Pub. No.: US 2009/0105668 A1
(43) Pub. Date: Apr. 23, 2009

(54) FEMININE VAGINAL DOUCHE FOR TIGHTENING A VAGINAL ORIFICE

(76) Inventors: Devon Monroe, Watertown, MA (US); Michael K. Hicks, Dorchester, MA (US)

Correspondence Address: BROWN RUDNICK LLP, ONE FINANCIAL CENTER, BOSTON, MA, 02111, US

(21) Appl. No.: 12/254,357

(22) Filed: Oct. 20, 2008

Related U.S. Application Data
(60) Provisional application No. 60/981,157, filed on Oct. 19, 2007.

(51) Int. Cl. A61M 31/00; A61F 5/00

(52) U.S. Cl. 604/279; 600/38

(57) ABSTRACT

A feminine vaginal douche and a method for simulating sexual intercourse. The feminine vaginal douche including a douche device, a dispenser, and a douche formula. The method includes the steps of inserting a vaginal douche into an orifice, dispensing douche formula into an orifice, and stimulating muscles of the orifice to expand and contract.

### CROSS-REFERENCE TO RELATED APPLICATIONS

This patent application claims priority to U.S. Patent Application Ser. No. 60/981,157, filed in the USPTO on Oct. 19, 2007 by Monroe et al., the entire contents of that application being incorporated herein by reference in its entirety.

### BACKGROUND

1. Technical Field

The present disclosure generally relates to the field of feminine vaginal douches, and more particularly, to a feminine vaginal douche that is used to tighten a vaginal orifice and simulate sexual intercourse.

2. Description of the Related Art

Feminine vaginal douches are used to cleanse, refresh, sooth, deodorize, and prevent skin irritations within a vaginal orifice. These vaginal douches involve an application of a liquid preparation contained in a douche device. The liquid preparations are typically comprised of one or more of the following: aluminum sulfate, water, sulfite, tin, other chemicals compositions, and the like.

Although feminine vaginal douches are used to provide relief to a vaginal orifice, these douches suffer from several drawbacks. For example, prior art douches are provided in various embodiments, such as suppositories, foams, creams, gels, ointments, enemas, and the like. As a result, these douches provide an uncomfortable and untidy application of the liquid preparation contained in the douche device. Further, prior art douches do not address the prevailing issue of looseness and lack of tightness in a vaginal orifice, which naturally occurs due to aging and/or lack of exercise. Additionally, feminine vaginal douches do not enhance sexual function or provide sexual stimulation.

Therefore, it may be beneficial to develop a formula to be used in a vaginal douche device, which may tighten a vaginal orifice as well as provide sexual stimulation.

### SUMMARY

According to an aspect of the present disclosure, a feminine vaginal douche is presented. The feminine vaginal douche is generally comprised of a douche device, a dispenser, and a douche formula. The douche device is provided for introduction into a vaginal orifice. The dispenser is in communication with the douche device and the douche formula is comprised of alum.

According to another aspect of the present disclosure, a method for simulating sexual intercourse is presented. The method includes inserting a vaginal douche into an orifice, dispensing a douche formula into the orifice, and stimulating muscles of the orifice to expand and contract. The method further includes a vaginal douche comprised of one or more of the following: a douche device, a douche formula, and a douche dispenser. Additionally, the method includes a douche formula comprised of at least one or more of the following: alum and water. Further, the step of stimulating results from astringent properties of the douche formula.

### BRIEF DESCRIPTION OF THE DRAWINGS

The objects and features of the present disclosure, which are believed to be novel, are set forth with particularity in the appended claims. The present disclosure, both as to its organization and manner of operation, together with further objectives and advantages, may be best understood by reference to the following description, taken in connection with the accompanying drawing as set forth below:

FIG. 1 is a perspective view of an embodiment of a feminine vaginal douche, according to the present disclosure.

### DETAILED DESCRIPTION OF EXEMPLARY EMBODIMENTS

The present disclosure generally relates to field of feminine vaginal douches, and more particularly, to a feminine vaginal douche that is used to tighten a vaginal orifice and simulate sexual intercourse. Reference will now be made in detail to exemplary embodiments of the present disclosure, which are illustrated in the accompanying figure. Additionally, the following detailed description does not limit the present disclosure.

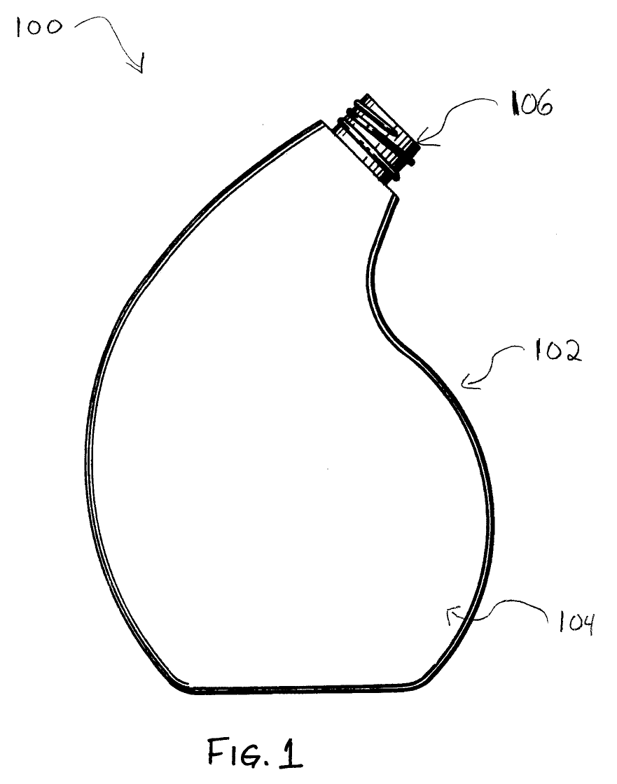

Referring to FIG. 1, a vaginal douche 100 is presented. Vaginal douche 100 is generally comprised of a douche device 102, a douche formula 104, and a dispenser 106. Douche device 102 is designed to provide a housing to douche formula 104. Further, douche device 102 is designed to disperse douche formula 104 through dispenser 106. In an embodiment of the present disclosure, dispenser 106 is affixed to the top portion of douche device 102. In a preferred embodiment, douche device 102 is provided as a bottle that contains douche formula 104. For example, douche device 102 may be, but is not limited to, a disposable bottle, such as 4.5 to 6.0 ounce bottle.

Douche formula 104 is a composition comprised of alum. Douche formula 104 may be, for example, a composition of water and alum. The alum of douche formula 104 may be aluminum sulfate. Further, douche formula 104 is generally comprised of a ratio of alum and water. For example, douche formula may be, but is not limited to, a ratio of 2.1 grams of aluminum sulfate per 133 milliliters of water. In an embodiment, the ratio of douche formula 104 is 0.5 teaspoons of aluminum sulfate per 4.5 ounces of water. In another embodiment, douche formula 104 may consist of a stronger or weaker formulation. Further, douche formula 104 may be formulated in any one or more of the following applications, such as creams, milks, gels, dispersions, micro-emulsions, lotions, ointments, sprays, pastes, jellies, aerosols, and the like. Douche formula 104 may also contain fragrances or flavors in other embodiments.

Dispenser 106 may be, but is not limited to, a tube that is affixed to douche device 102. For example, dispenser 106 may be a plastic tube that is temporarily or permanently affixed to douche device 102. In an embodiment, dispenser 106 is provided as a tube with a screw-like mechanism, which provides a convenient method to remove dispenser 106 from douche device 102.

In another embodiment of the present disclosure, a method for utilizing vaginal douche 100 for simulating sexual intercourse is provided. Douche device 102 may be presented having douche formula 104, comprised of alum, stored therein. Dispenser 106 may be affixed to the top portion of douche device 102 and inserted into an orifice. The orifice may be, for example, a vaginal or anal orifice.

Douche formula 104 may be dispensed from douche device 102 through dispenser 106. The step of dispensing may be performed by applying pressure to the housing of douche device 102. For example, a plastic bottle may be manipulated to easily dispense douche formula 104.

Further, the step of dispensing douche formula 104 into an orifice may simulate sexual intercourse. For example, douche formula 104 may cause a vaginal orifice to tighten upon contacting the walls of the vaginal orifice. In an embodiment, douche formula 104 acts as an astringent and may cause the muscles of the vaginal wall to expand and contract. In another embodiment, the application of douche formula 104 into an orifice, such as a vaginal or anal orifice, may be used as a sexual aid by means of causing the muscles to contract to simulate each partner during sexual intercourse.

It will be understood that various modifications may be made to the embodiments disclosed herein. Therefore, the above description should not be construed as limiting, but merely as exemplifications of the various embodiments of the invention. Those skilled in the art will envision other modifications within the scope and spirit of the claims appended hereto.

What is claimed is:

1. A feminine vaginal douche, comprising:
a douche device for introduction into a vaginal orifice;
a dispenser in communication with the douche device; and
a douche formula comprising alum.

2. The feminine vaginal douche of claim 1, wherein the douche formula includes water.

3. The feminine vaginal douche of claim 1, wherein the alum is substantially a derivative of aluminum sulfate.

4. The feminine vaginal douche of claim 1, wherein a ratio of the douche formula is 2.1 grams of aluminum sulfate per 133 milliliters of water.

5. The feminine vaginal douche of claim 1, wherein a ratio of the douche formula is 0.5 teaspoons of aluminum sulfate per 4.5 ounces of water.

6. The feminine vaginal douche of claim 1, wherein the douche device is disposable.

7. The feminine vaginal douche of claim 1, wherein the dispenser is detachable from the douche device.

8. A method for simulating sexual intercourse, comprising the steps of:
inserting a vaginal douche into an orifice, the vaginal douche comprised of one or more of the following: a douche device, a douche formula, and a douche dispenser;
dispensing the douche formula into the orifice, the douche formula comprising at least one or more of the following: alum and water; and
stimulating muscles of the orifice to expand and contract, the step of stimulating resulting from astringent properties of the douche formula.

9. The method of claim 8, wherein the orifice is a vaginal orifice.

10. The method of claim 8, wherein the orifice is an anal orifice.

11. The method of claim 8, wherein the alum is substantially a derivative of aluminum sulfate.

12. The method of claim 8, wherein a ratio of the douche formula is 2.1 grams of aluminum sulfate per 133 milliliters of water.

13. The method of claim 8, wherein a ratio of the douche formula is 0.5 teaspoons of aluminum sulfate per 4.5 ounces of water.
